(19) United States
(12) Patent Application Publication
(10) Pub. No.: US 2010/0184190 A1
(43) Pub. Date: Jul. 22, 2010

(54) Influencing viral lipid constituents

(75) Inventor: Vu Truong-Le, Campbell, CA (US)

Correspondence Address: QUINE INTELLECTUAL PROPERTY LAW GROUP, P.C., P O BOX 458, ALAMEDA, CA, 94501, US

(73) Assignee: MedImmune, LLC

(21) Appl. No.: 12/311,757

(22) PCT Filed: Oct. 9, 2007
(86) PCT No.: PCT/US07/21674

§ 371 Date: Mar. 15, 2010

Related U.S. Application Data
(60) Provisional application No. 60/852,571, filed on Oct. 17, 2006.

(51) Int. Cl. C12N 7/00

(52) U.S. Cl. 435/235.1

(57) ABSTRACT

This invention provides compositions, methods and systems to modulate the lipid content and membrane characteristics of cells and virions. Growth of host cells on media containing particular amounts, classes and/or combinations of lipid supplements can influence the lipid content of the cell and viruses grown on the cell. Lipids, such as cholesterol esters, sphingomyelin, glycolipids, containing C16:0, C18:0, C18: 1n9 and/or C18:2n6 fatty acids, can influence cell permissivity for virus infection, virus yield, virus immunogenicity and/or membrane phase transition temperatures.

### CROSS-REFERENCE TO RELATED APPLICATIONS

This application claims priority to and benefit of a prior U.S. Provisional Application No. 60/852,571, Influencing Viral Lipid Constituents, by Vu Truong-Le, filed Oct. 17, 2006. The full disclosure of the prior application is incorporated herein by reference.

### FIELD OF THE INVENTION

The inventions involve compositions of media, cells and viruses having modified lipid constituents. Methods to provide the cells and viruses include adjustment of media lipids, such as sphingomyelin, cholesterol, phosphatidylserine and phosphatidylcholine, for culture of virus host cells. Modified host cells can be more permissive and higher yielding for culture of the virus. Resultant modified viruses can be more stable and better suited to conditions of processing.

### BACKGROUND OF THE INVENTION

Cell plasma membranes contain a variety of lipids (e.g., phospholipids, cholesterol, sphingolipids, and glycolipids), but they are not necessarily uniformly distributed. For example, significant differences are encountered between the lipid constituents of internal and external facing layers of the membrane bilayer. Moreover, lipids and certain membrane proteins can form “rafts” or lipid microdomains with lipids more tightly packed than in the membrane generally, e.g., due to enrichment for specific lipids having more highly saturated fatty acids. The lipid composition of membranes can substantially affect the characteristics of the membranes.

Culture media may influence the lipid composition of animal cell membranes. For example, in D. Tombaccini, et al., Lipid Composition of Balb/c3T3, SV3T3, and Conconavalin A-Selected Revertant Cells Grown in Media Containing Lipid-Depleted Serum, J. Lipid Res., 22: 590-597, (1981), a variety of changes to membrane lipid fatty acid composition were noted when cells were grown under stress in lipid-depleted media. Many of these changes were similar to those found in tissues of animals kept on fatty acid deficient diets. However, it has not been clear that supplementation of normal animal feed or cell culture media would provide significant alterations from normal membrane lipid composition.

Attempts have been made to affect the lipid composition of enveloped viruses. For example, A. C. Roland, et al., “Lipid Composition and Fluidity of the Human Immunodeficency Virus Envelope and Host Cell Plasma Membranes”, PNAS 90: 5181-5185 (1993), found that there were differences between the HIV envelope and host cell membranes. Roland notes that incubation of HIV-1 with a phospholipid liposome reduced the amount of cholesterol in the envelopes, resulting in a significant drop in infectivity.

However, a need remains for more selective methods to influence the lipid composition of host cells and viruses. Identification of particular lipids and constituent fatty acids associated with normal infection and replication may enable benefits in culture and control of certain viral characteristics. The present invention provides these and other features that will become apparent upon review of the following.

### SUMMARY OF THE INVENTION

The present invention involves compositions, methods and systems for modulating the lipid composition of cell membranes and virus envelopes. Host cells can be grown in media containing certain lipids that are supplemented to influence the lipid class and subclass provided in the cell membrane. The media used to grow virus host cells can influence the character of lipids in the virus envelope, affect the virus yield and influence physical properties of the virus.

Methods of modifying a virus lipid composition can include, e.g., modulating levels of sphingomylein or cholesterol in a culture media for culturing a host cell suitable for the virus, and infecting the host cell with the virus. Thereby, progeny virions can be provided with a different lipid composition than for the same (control) virus grown on the same host cells without the modulation of these media lipid concentrations. Typical viruses for modulation by the present methods include, e.g., retrovirus (such as HIV), paramyxovirus (such as respiratory syncytial virus, parainfluenza virus, metapneumovirus), orthomyxovirus (such as influenza virus), papovavirus (such as human papilloma virus), filovirus, poxvirus, herpesvirus, hepadnavirus, rhabdovirus (such as rabies), coronavirus (such as SARS), togavirus, arenavirus, and bunyavirus.

Modulation of media can include increasing or decreasing a standard media concentration of lipids identified herein. Modulating can include preparing a media of defined or unknown lipid content, then modulating the media lipids by changing the lipid content of the media (typically, by adding defined classes and/or subclasses of lipids to the media). Unmodulated cells can be grown on the unmodulated starting media, whereas modulated cells can be grown on the modulated media. Modulated virions can be cultured in the modulated cells, whereas unmodulated virions are grown on the unmodulated cells. For example, levels of the lipids in a media can be modulated levels different from starting media, e.g., to greater than 20 mg/L or less than 0.01 mg/L in the media. The methods include modulation of lipids in the classes of sphingomylein, cholesterol, phospholipids, and glycolipids. Further, modulation of lipids can include increasing or decreasing subclasses of lipids esterified to certain fatty acids, such as, e.g., 9-octadecenoic acid (C18:1n9), palmitate (C16:0), stearate (C18:0), linoleic acid (C18:2n6) and oleate (C18:1).

The modulation of virus lipid composition in this fashion can change certain physical properties of the virus such as the viral membrane phase transition temperature. A viral membrane phase transition temperature can thus be adjusted to affect viral storage stability or to compliment desired formulation processes parameters such as drying temperatures, e.g., to aid in penetration of preservative molecules into the virus. For example, the modulated virus can be made with a specific phase transition temperature of the lipid such that when it is held at the transition temperature in a liquid preservative formulation, transmembrane uptake of stabilizers can be influenced before and/or during drying processes for preparation of a vaccine dry formulation. Exemplary drying methods can include freeze drying, freeze foam drying, foam drying, spray freeze drying, spray drying, high pressure spray drying, and supercritical spray drying.

Modulation of a virus envelope lipid content can be used to influence the immunogenicity of the virus. The virus envelope externally presented to the host organism immune system, so is important in immune interactions. Combinations of membrane lipids and proteins forming “lipid rafts” on the envelope can be significant immunogens. Depending on the membrane lipid composition, the overall protein antigenic conformation and presentation of immunogens can be affected. For example, a viral vaccine for a mammal but grown in a bird can be adjusted to elicit a more protective response, e.g., by modifying the virus to have a more typical mammalian lipid content. Optionally, the lipid content of a virus could be adjusted to elicit a more intense and lasting immune response, e.g., an adjuvant effect.

In another aspect, the permissivity of the host cells to infection can be influenced by modulating levels of certain lipids in the growth media. For example, host cells can be grown in media with added or supplemental lipids correlated to permissivity. With regard to Influenza viruses and others, infectivity of host cells can be enhanced by growing them in media enriched with sphingomylein and/or cholesterol. It can be particularly beneficial to include lipid subclasses with C18:0, C18:1n9 and/or C18:2n6 fatty acids in the media.

In still another aspect of the invention, the yield of viruses can be increased, e.g., by providing media enriched for lipids selectively incorporated into the virus envelope. For example, the production of a virus could be increased from a cell by modulating the levels of sphingomylein or cholesterol in the growth media for the cell. Optionally, the virus yield could be increased by adding phosphatidylserine or phosphatidylinositol to the media. It could be particularly beneficial to include lipid subclasses with C16:0, and/or C18:2n6 fatty acids in the media.

The invention includes compositions of an Influenza virion having an increased proportion of membrane cholesterol or sphingomylein as compared to a control influenza virion grown in the allantoic sac of a chicken egg or cultured cell lines expressing less than optimal concentrations of such preferred lipids. In certain cases, the increased proportions can result from culture of the virion on host cells grown in media containing 20 mg/L or more of sphingomylein or cholesterol. In preferred embodiments, sphingomylein esterified to palmitate (C16:0) is included in the media and/or cholesterol is esterified to palmitate (C16:0) or stearate (C18:0) is included in the media. In more preferred embodiments, the mole percentage fatty acids in the media sphingomylein palmitate (C16:0) is increased by at least 10% over that of a standard media or the cholesterol palmitate (C16:0) or stearate (C18:0) is increased by at least 10% over that of the standard (e.g., starting) media. In more preferred embodiments, the mole percentage fatty acids in membrane sphingomylein palmitate (C16:0) is increased by at least 10% over that of the control virion or the cholesterol palmitate (C16:0) or stearate (C18:0) is increased by at least 10% over that of the control virion (e.g., from cells infected and grown in standard media).

Systems for culture of a virus include, e.g., a host cell permissive for the virus, the cell in culture media suitable for growing the cell, and cholesterol or sphingomylein. Addition of the cholesterol or sphingomylein to the media can be intended to modulate the permissivity, increase yield of the virus from the cell, increase stability of the virus in storage or change the membrane phase transition temperature of the virus. In preferred embodiments, the added cholesterol or sphingomylein comprise more than 25 mole percent palmitate (C16:0), more than 20 mole percent stearate (C18:0) or more than 40 mole percent oleate (C18:1). In preferred systems, the virus is a retrovirus (such as HIV), paramyxovirus (such as respiratory syncytial virus, parainfluenza virus, metapneumovirus), orthomyxovirus (such as influenza virus), papovavirus (such as human papilloma virus), filovirus, poxvirus, herpesvirus, hepadnavirus, rhabdovirus (such as rabies), coronavirus (such as SARS), togavirus, arenavirus, and bunyavirus. In preferred embodiments, the host cell is a chicken egg allantoic sac cell or MDCK kidney cell. In embodiments, e.g., to increase virus production, the media also includes phosphatidylserine and/or phosphatidylinositol.

In certain methods of the invention, the lipid content of virus host cells are modulated by adjusting the diet of the animal providing the host cells. For example, an Influenza virus with a modified membrane lipid composition could be provided by feeding a chicken a diet rich in cholesterol or sphingomylein (or other desired lipid classes and/or subclasses), collecting one or more fertile eggs from the chicken, inoculating the allantoic sacs of the one or more eggs with an influenza virus, and harvesting influenza viruses from the one or more allantoic sacs. In preferred embodiments, chicken feed is enriched until the allantoic fluid total cholesterol and/or sphingomylein comprises more than 25 mole percent palmitate (C16:0), more than 20 mole percent stearate (C18:0) and/or more than 40 mole percent oleate (C18:1). In preferred embodiments the diet rich in cholesterol or sphingomylein comprises at least 0.5 grams, 1 gram, 2 grams, 5 grams or 10 grams of total cholesterol or total sphingomylein per chicken per day. The viruses thus provided can have a desired characteristic, such as, e.g., a higher titer than for virus cultured on eggs from control chickens not fed a diet relatively enriched in the modulated lipid, a longer shelf life than for virus cultured on eggs from control chickens not fed a diet enriched in the modulated lipid, a higher antigenicity than for virus cultured on eggs from control chickens not fed a diet enriched in the modulated lipid, and a modified membrane phase transition compared to a membrane from a virus cultured on eggs from control chickens not fed a diet enriched in the modulated lipid.

### DEFINITIONS

Unless otherwise defined herein or below in the remainder of the specification, all technical and scientific terms used herein have meanings commonly understood by those of ordinary skill in the art to which the present invention belongs. The following definitions supplement those in the art and are directed to the current application and are not to be imputed to any related or unrelated case, e.g., to any commonly owned patent or application.

Before describing the present invention in detail, it is to be understood that this invention is not limited to particular devices or systems, which can, of course, vary. It is also to be understood that the terminology used herein is for the purpose of describing particular embodiments only, and is not intended to be limiting.

As used in this specification and the appended claims, the singular forms “a”, “an” and “the” optionally include plural referents, unless the content clearly dictates otherwise. Thus, for example, reference to “a lipid” can include a combination of two or more lipids; reference to “eggs” can include a single egg, and the like.

Although many methods and materials similar, modified, or equivalent to those described herein can be used in the practice of the present invention without undue experimentation, the preferred materials and methods are described herein. In describing and claiming the present invention, the following terminology will be used in accordance with the definitions set out below.

A class of membrane lipids includes named lipid molecule types that can exist as certain sub classes, depending, e.g., on what fatty acids are esterified to the lipid. For example, classes of membrane lipids can include: cholesterols, sphingomyelins, phospholipids, glycolipids, phosphatidylcholines, phosphatidylserines, phosphatidylinositols, lysophosphatidylcholines, cardiolipins, phosphatidylethanolamines, ceramides, cerebrosides, gangliosides, and the like. Subclasses of these lipids can include class members esterified to different fatty acids, such as, e.g., myristic acid, palmitic acid, stearic acid, oleic acid, linoleic acid, linonenic acid, arachidonic acid, eicosapentaenoic acid, and/or the like. A virus has a “different lipid composition”, compared to another virus, if one or more lipid class or subclass is present in the virus in an amount or proportion significantly (e.g., 10% difference) different from the other virus. The present invention provides methods to produce viruses with different lipid compositions, e.g., starting with the same inocula, but culturing on cells grown on media with different lipid ingredients and/or different lipid proportions.

Fatty acid chain short hand notations, such as C16:0, C18:1n9, are as commonly known in the field. For example, the notation C18:1 indicates an 18 carbon fatty acid chain having a 1 double bond at an unsaturated position in the chain. C18:1n9 indicates an 18 carbon fatty acid chain having a double bond at the 9^{th} carbon. Typically, the fatty acids are esterified to a more polar group in the lipid molecule to form, e.g., a phospholipid, sphingolipid, glycolipid, or the like. In other cases, the fatty acids can be esterified to a relatively hydrophobic group, such as cholesterol.

As used herein, the term “modulating” with regard to lipids refers to changing the amount, concentration and/or proportions of lipids, lipid classes and/or lipid subclasses. In many cases, modulating, e.g., media lipids entails addition of lipid to a starting media. However, modulating can optionally include reducing or eliminating a lipid from a media, cell membrane or virus.

### BRIEF DESCRIPTION OF THE DRAWINGS

FIG. 1 shows charts of FTIR data for cell membrane lipids. Control chicken embryo kidney (CEK) cells demonstrate phase transitions at about 7° C., 32° C. and 54° C. (chart 1A). Removal of cholesterols from the CEK cell membranes using cyclodextrin caused a significant increase in about 45° C. melting lipid ordered groups (1B) thus indicating that plasma membranes of CEK cells, which are strongly permissive to Influenza infection, contain significant amounts of cholesterol and cholesterol esters. Treatment of the membranes with sphingomyelinase results in significant reduction in the lower melting groups and a large increase in more ordered high melting membrane lipid groups. This response strongly suggests that CEK cells contain a significant amount of sphingomyelin. Taken together, the data suggested that cholesterol and sphingomyelin are significant components of CEK membranes and suggest that these lipids contribute to Influenza infection permissivity.

FIG. 2 shows a chart of FTIR data for A/Sydney virus envelope lipids. Treatment with cyclodextrins to reduce cholesterol content, or with sphingomyelinase to convert sphingomyelin to ceramide, results in significant increases in ordered high melting membrane lipid groups. The data demonstrate that, like CEK cells, an Influenza virus such as A/Sydney contains a significant amount of cholesterols and sphingolipids.

FIG. 3 shows a histogram chart comparing representation of certain lipid classes in membranes of chicken allantoic sac (CAS) host cell and Influenza viruses produced from on CAS cells that lined the egg allantoic sacs. Compared to typical distribution of various lipid classes from common mammalian cells, the data suggest an enrichment in phosphatidylserine and sphingomyelin in Influenza viruses as well as CEK cells, which are known to be very permissive to influenza infection.

FIGS. 4 and 5 show histogram charts comparing representation of phospholipid subclasses between CAS cells and viruses grown on CAS cells.

FIG. 6 shows a histogram chart comparing representation of sphingomyelin subclasses between CAS cells and viruses grown on CAS cells.

FIG. 7 shows a histogram chart comparing representation of cholesterol ester subclasses between CAS cells and viruses grown on CAS cells. The results suggest that significant lipid composition differences exist between permissive hosts permissive hosts.

FIG. 8 shows membrane lipid class differences between viruses and between cells having different levels of permissivity for the viruses. The results suggest that significant lipid composition differences exist between permissive hosts and non-permissive hosts.

FIG. 9 shows differences in membrane cholesterol ester subclasses between viruses and cells having different levels of permissivity for the viruses.

FIG. 10 shows differences in membrane sphingomyelin subclasses between viruses and cells having different levels of permissivity for the viruses.

### DETAILED DESCRIPTION

The present inventions involve viruses with modified lipid composition and methods to influence the lipid make up of viruses and their host cells. In one aspect, the cell membrane lipids of virus host cells can be modified, e.g., by adjustment of media constituents. Such modified host cells can be infected with increased permissivity and provide increased yields of viruses. The viruses cultured in the modified cells can have, e.g., modified viral membrane lipid compositions providing enhanced stability, modified immunogenicity, and/or changed membrane phase transition characteristics, compared to the same virus grown on unmodified host cells.

In particular embodiments of the methods, media can be provided comprising cholesterol, sphingomyelin, phosphatidylserine/inositol and/or phosphatidylcholine in amounts that affect the lipid composition of a host cell membrane (external or internal) and/or the lipid (e.g., viral membrane) composition. The resultant membrane compositions can provide benefits in the culture, processing parameters, stability and antigenic character of viruses cultured from cells grown in the media.

We have found that significant lipid composition differences exist between permissive hosts and non-permissive hosts, and between viruses and their host cells, that could be taken advantage of, e.g., to provide virus vaccines having more potency, stronger immunogenicity, more convenient processing, and/or longer shelf life. For example, cells modified to have increased membrane sphingomyelin (particularly with a higher proportion of 18:0 (stearic) fatty acid chains) and/or cholesterol esters (particularly with 18:1n9 (oleic) fatty acid chains) can have increased permissivity for viruses, such as, e.g., retroviruses (such as HIV), paramyxovirus (such as respiratory syncytial virus, parainfluenza virus, metapneumovirus), orthomyxovirus (such as Influenza virus), papovavirus (such as human papilloma virus), filovirus, poxvirus, herpesvirus, hepadnavirus, rhabdovirus (such as rabies), coronavirus (such as SARS), togavirus, arenavirus, and bunyavirus. Optionally, the lipid content of a virus can be effected by the composition of host cell media, to provide a higher proportion of virus preferred lipids and fatty acids, such as C16:0 cholesterol esters, phosphatidylserine, phosphatidylinositol and C16:0 (palmitic) sphingolipids and 18:2n6 (linoleic) cholesterol.

### Influencing Lipids of a Host Cell

In light of the correlations provided herein between lipid content and certain host cell characteristics, and considering the fact that the lipid composition of host cells can be influenced by the lipid content of growth media, strategies are provided herein to improve characteristics of virus host cells.

In one aspect, culture conditions for virus host cells can be adjusted to enhance the permissivity to infection and to improve the ultimate virus culture yield. It has been found that the permissivity of infection, e.g., for host cells to Influenza viruses, correlates to higher proportions of C18:1n9 cholesterol in the host cell lipid membranes. We have also found a trend of increased C18:0 sphingomylein in the membrane lipids of more permissive host cells. In methods of the invention, virus host cells are grown in media containing C18:1n9 cholesterol esters and/or C18:0 sphingomylein. In methods of the invention, virus host cells are grown in media containing 20 mg/L, 30 mg/L, 50 mg/L, 100 mg/L, 500 mg/L, or more of C18:1n9 cholesterol esters and/or C18:0 sphingomylein.

In certain embodiments of the invention, the amount of C18:1n9 cholesterol esters and/or C18:0 sphingomylein is modulated in culture media for virus host cells. For example, various amounts of C18:1n9 cholesterol esters and/or C18:0 sphingomylein can be added to a standard commercial media (or any other starting media) to ensure the presence of these lipids, increase the absolute amount of these lipids, and/or to increase the proportion of these lipids. Optionally, media can be formulated with less, or a lower proportion of other cholesterol esters, such as C16:0 and/or C18:0 cholesterol esters, than for C18:1n9 cholesterol esters. Optionally, host cell culture media can be formulated without or with less or a lower proportion of other sphingolipids, such as C16:0 and/or C18:1n9 sphingomyelin, than for C18:0 sphingomyelin. For example, host cells for culture of enveloped viruses can be grown in media containing C18:1n9 cholesterol esters in excess over other individual cholesterol esters, or in excess over a combination of other cholesterol esters. Optionally, host cells for culture of enveloped viruses can be grown in media containing C18:0 sphingomylein in excess over other individual sphingolipids, or in excess over a combination of other sphingolipids. In one embodiment, for example, a liquid media for culture of enveloped virus host cells contains at least 1 mg/L, 5 mg/L, 10 mg/L, 25 mg/L, 50 mg/L, 100 mg/L, 200 mg/L, 400 mg/L, or more of C18:1n9 cholesterol esters. In this embodiment, it is preferred that cholesterol esters that do not comprise C18:1n9 fatty acid be absent from the culture media, be present in individual amounts less than for C18:1n9 cholesterol esters, or be present in total combined amounts less than for C18:1n9 cholesterol esters. In certain embodiments, a liquid media for culture of enveloped virus host cells contains at least 1 mg/L, 5 mg/L, 10 mg/L, 25 mg/L, 50 mg/L, 100 mg/L, 200 mg/L, 400 mg/L, or more C18:0 sphingomylein. In these embodiments, it is preferred that sphingomyelin that does not comprise C18:0 fatty acid be essentially absent from the culture media, be present in individual amounts less than for C18:0 sphingomyelin, or be present in total combined amounts less than for C18:0 sphingomyelin. In another aspect, it is preferred that C18:1n9 cholesterol esters be present in culture media for enveloped virus host cells in amounts at least 35 mole percent, at least 40%, at least 50%, at lease 60%, or more of the total constituent cholesterol esters of the media. Further, it is preferred that C18:0 sphingomyelin be present in culture media for enveloped virus host cells in amounts at least 15 mole percent, at least 25%, at least 50%, at lease 60%, or more of sphingolipids in the media.

In other aspects of the compositions and methods of the invention, we have found that permissivity of host cells to envelope viruses is higher when the cells or media lipids comprise C18:2n6 fatty acids. These fatty acids can be free fatty acids, or esterified in membrane lipid molecules, such as C18:2n6 cholesterol, C18:2n6 cholesterol esters, and/or C18:2n6 sphingomyelin. Preferred liquid media for culture of enveloped virus host cells, contains at least 0.5 mg/L, 1 mg/L, 5 mg/L, 10 mg/L, 25 mg/L, 50 mg/L, 100 mg/L, 200 mg/L, or more of C18:2n6 fatty acids or lipids containing these fatty acids. In another aspect, it is preferred that C18:2n6 cholesterol esters be present in culture media for enveloped virus host cells in amounts at least 5 mole percent, at least 10%, at least 15%, at lease 20%, or more of total constituent cholesterol esters. Optionally, C18:2n6 sphingomyelin can beneficially be present in culture media for enveloped virus host cells in amounts at least 5 mole percent, at least 10%, at least 15%, at lease 20%, or more of total constituent sphingolipids of the media.

In a most preferred embodiment, the permissivity of host cells for virus infection is modulated by supplementation of the growth media with a combination of the lipids discussed above. For example, in a preferred embodiment, the host cell growth media is supplemented with two or more of: C18:1n9 cholesterol esters, C18:0 sphingomylein, C18:2n6 sphingomyelin and C18:2n6 cholesterol. In one aspect, the media is supplemented to provide at least 35 mole percent of cholesterol esters being C18:1n9 esters and at least 15 mole percent of sphingolipid fatty acids being C18:0 fatty acids. In another aspect, the media is supplemented to provide at least 35 mole percent of cholesterol esters being C18:1n9 esters, at least 15 mole percent of sphingolipid fatty acids being C18:0 fatty acids, and at least 5 mole percent of sphingolipids fatty acids being C18:2n6 fatty acids.

### Influencing Virus Titer

The virus titer from a culture can strongly affect the time and expense of preparing and processing the virus harvest. Methods of the present invention can improve the productivity, e.g., of enveloped virus culture by providing lipids correlated to enhanced host cell permissivity to infection by the virus and/or by providing lipids selectively enriched in the lipids of the virus over those of the host.

Viruses benefiting from the methods of the invention generally include the enveloped viruses. However, the present invention recognizes that the permissivity of host cells for non-enveloped viruses can benefit from the media lipid adjustments described herein. Furthermore, we recognize that many non-enveloped viruses have lipid and/or lipoprotein components that can benefit from the present media modifications, e.g., to provide more stable and/or more productive virion progeny.

Preferred viruses for employment in the compositions, methods and systems of the invention include the enveloped viruses. In particular, more preferred viruses that can benefit from culture on cells grown in the inventive media are, e.g., members of the Baculoviridae, Herpesviridae, Iridoviridae, Poxviridae, Cystoviridae, Coronaviridae, Flaviviridae, Togaviridae, Arterivirus, Retroviridae, Orthomyxoviridae, Filoviridae, Paramyxoviridae, Rhabdoviridae, Arenaviridae, and Bunyaviridae families. Most preferred viruses include the various Influenza viruses, human immunodeficiency (HIV) viruses, paramyxovirus (such as respiratory syncytial virus, parainfluenza virus, metapneumovirus), and papovavirus (such as human papilloma virus).

With regard to increasing virus titer using methods of the invention, this can be accomplished according to the methods described above for increasing the permissivity of the host cell. Permissivity of a cell for infection by a virus is a measure of whether, and to what extent, the cell can act as a host to foster replication of the virus. The virus can not replicate in a cell that is not permissive for the virus, for whatever reason. On the other hand, cells that are more permissive for a virus, will typically replicate a higher number of the virions per cell than for less permissive cells. Another benefit of a more permissive cell is that even if the progeny virions per cell is not high, at least the efficiency of infection can be high. That is, more permissive cells can often become infected by a higher proportion of inocula virions, e.g., so that infection with a low titer or degraded inoculum can be more efficiently propagated. Therefore, it is an aspect of the invention that methods of the invention to increase permissivity of host cells can be used to enhance the titer of viruses obtained from infection of the host cell culture.

In another aspect of the invention, the quality and/or quantity of virus produced from infection of host cells can be influenced by provision of lipids that are relatively abundant in the virus or relatively enriched in the virus over the host cell lipids. For example, we have determined that a virus can have a lipid content relatively high in certain lipids, as compared to the host cell media and/or the host cell. Provision of the lipids, selectively enriched in the virus, is expected to provide benefits in culture of the virus, e.g., enhanced permissivity, increased infectivity, higher titers, and/or higher yield.

We have found that certain enveloped virus particles are enriched for lipids containing C16:0 fatty acids. In one embodiment, the productivity of a virus culture can be increased by addition of C16:0 fatty acids, or lipids comprising C16:0 fatty acids, in the media used to culture the host cell for the virus. For example, in certain embodiments, a virus can be cultured on host cells grown on a liquid media containing at least 1 mg/L, 5 mg/L, 10 mg/L, 25 mg/L, 50 mg/L, 100 mg/L, 200 mg/L, 400 mg/L, or more of a C16:0 fatty acid (palmitic acid), its salts, or a lipid comprising C16:0 fatty acid, such as a cholesterol ester or a sphingolipid. In these embodiments, it is preferred that C16:0 lipids listed above be present in amounts greater than for other fatty acids of individual types of lipids or for the lipids in total. In another aspect of the invention, it is preferred that C16:0 cholesterol esters be present in culture media for enveloped virus host cells in amounts at least 10 mole percent, at least 15%, at least 20%, at lease 30%, at least 40% or more of the total media cholesterol esters. Further, it is preferred that C16:0 sphingomyelin be present in culture media for enveloped virus host cells in amounts at least 10 mole percent, at least 15%, at least 20%, at lease 30%, at least 40% or more of the media sphingolipids.

We have found that certain enveloped virus particles are enriched for lipids containing C18:2n6 fatty acids. In one embodiment, the productivity of a virus culture can be increased by addition of C18:2n6 fatty acids, or lipids comprising C18:2n6 fatty acids, in the media used to culture the host cell for the virus. For example, in certain embodiments, a virus can be cultured on host cells grown on a liquid media containing at least 1 mg/L, 5 mg/L, 10 mg/L, 25 mg/L, 50 mg/L, 100 mg/L, 200 mg/L, 400 mg/L, or more of a C18:2n6 fatty acid (linoleic acid), its salts, or a lipid comprising one or more C18:2n6 fatty acid, such as a cholesterol ester or a sphingolipid. In these embodiments, it is preferred that C18:2n6 lipids listed above be present in amounts greater than for other fatty acids of individual types of lipids or for the lipids in total. In another aspect of the invention, it is preferred that C18:2n6 cholesterol esters be present in culture media for enveloped virus host cells in amounts at least 10 mole percent, at least 15%, at least 20%, at lease 30%, at least 40% or more of the media cholesterol esters. Further, it is preferred that C18:2n6 sphingomyelin be present in culture media for enveloped virus host cells in amounts at least 2 mole percent, at least 5%, at least 10%, at lease 20%, at least 30% or more of the media sphingolipids.

In certain embodiments of the invention, the productivity (e.g., infectivity, replication, titer, infectious units per host cell) can be increased by growth of the virus host cell on media supplemented with a combination of the lipids cited above. For example, in a preferred embodiment, the virus host cell growth media is supplemented with two or more of: C16:0 cholesterol esters, C16:0 sphingomylein, C18:2n6 sphingomyelin and C18:2n6 cholesterol esters. In one aspect, the media is supplemented to provide at least 15 mole percent of cholesterol esters and/or sphingolipid fatty acids comprising C18:2n6 fatty acids. In another aspect, the media is supplemented to provide at least 10 mole percent of sphingolipid fatty acids being C16:0 fatty acids, and at least 15 mole percent of the cholesterol esters comprising C16:0 fatty acids. In another aspect, the media is supplemented to provide at least 10 mole percent of cholesterol esters being C18:2n6 esters, at least 10 mole percent of sphingolipid fatty acids being C18:2n6 fatty acids, at least 10 mole percent of cholesterol esters being C16:0 esters, and at least 5 mole percent of sphingolipid fatty acids being C16:0 fatty acids.

### Enhanced Virus Stability

Adjustment of the quantity and/or proportions of virus lipid constituents, according to the methods of the invention, can be used to improve aspects of the virus physical stability. For example, virus adjusted to contain more high melting lipids, lipids more complimentary to vaccine formulation processes, or lipids less prone to oxidation, can provide greater stability in certain environments, e.g., shelf life stability for vaccine products and/or processing intermediates.

The melting point of lipids is generally higher for those containing longer fatty acid chains and for those containing saturated fatty acids. In addition, the fluidity of membranes is decreased with increased proportions of cholesterol, therefore membranes with higher cholesterol content are more rigid and can be more stable. Moreover, the sensitivity of saturated fatty acid chains to oxidation (e.g., radical mediated chain reactions in storage) is less than for unsaturated fatty acids. In an aspect of the invention, the stability of viruses, particularly enveloped viruses, can be increased by culture on cells grown in media containing lipids rich in saturated fatty acids and/or low in cholesterol. For example, a media for culture of cells to produce more stable virions can include supplemental amounts of sphingomyelin and phospholipids comprising higher proportions of saturated fatty acids than standard media. Standard media (e.g., commercially available media) can be supplemented, e.g., with lipids with unusually high proportions of, e.g., C16:0 fatty acids, C18:0 fatty acids, and/or C20:0 fatty acids. In a preferred embodiment, growth media for virus host cells includes at least 1 mg/L, 5 mg/L, 10 mg/L, 25 mg/L, 50 mg/L, 100 mg/L, 200 mg/L, 400 mg/L, or more of lipids comprising one or more saturated fatty acid, such as, e.g., a C16:0 fatty acid, a C18:0 fatty acid, and/or C20:0 fatty acid. In these embodiments, it is preferred that the saturated C16-C20 fatty acids be present in amounts greater than for other fatty acids in particular lipids (e.g., phospholipid, sphingolipid, glycolipid, etc.) or for total lipids in the media. In another aspect of the invention, it is preferred that unsaturated C16-C20 fatty acids be present in culture media for enveloped virus host cells in amounts at least 10 mole percent, at least 15%, at least 20%, at lease 30%, at least 40% or more of an individual media lipid type or of the total media lipids.

We have noted (see application Ser. No. 10/412,630, incorporated herein by reference) that stability of biological materials (such as bacteria, enveloped viruses, animal cells, etc.) can be improved by exposing the membranous biologic to liquid preservative solutions at a temperature near the phase transition temperature of the membrane. At the phase transition temperature, the permeability of the membrane is substantially increased, thus allowing preservative materials (such as, standard biologic formulation sugars and amino acids) to more readily penetrate the membrane. It is an aspect of the invention that to render cells and/or viruses more suitable for formulation and preservation processes, the phase transition temperature of the cells or virus' host can modulated by adjustment of the media lipid content. For example, a typical phase transition temperature for a membrane can be about 15° C. However, it may be desirable to process the virus (e.g., vaccine) at a lower temperature or 5-10° C. to enhance viability through the process and/or to conform to temperatures encountered as a result of latent heat during, e.g., spray drying, lyophilization or freeze-foam drying. On the other hand, it may be desirable to modulate the phase transition temperature of a cell membrane to a higher temperature, e.g., for membranes with inherently low phase transition temperatures (e.g., psychrophilic life forms) or processes where membrane permeability at higher temperatures is desired (e.g., generic transformation processes).

In general, the rigidity and phase transition temperature of membranes can be increased by, e.g.: 1) growing the cell in media deficient in cholesterol, 2) growing the cell in media supplemented with sphingomyelin, 3) exposing the cells to cyclodextrin (to reduce the membrane cholesterol), 4) exposing the membrane to sphingomyelinase (thus, converting membrane sphingomyelin to more rigid ceramide), and/or 5) growing the cell in media supplemented with lipids comprising fatty acids having a high proportion of long chain and/or saturated fatty acids. In general, the phase transition temperature of membranes can be lowered by, e.g.: 1) growing the cell in media supplemented with Cholesterol, 2) growing the cell in media deficient in sphingomyelin, and/or 3) growing the cell in media supplemented with lipids comprising fatty acids having a high proportion of short chain and/or unsaturated fatty acids.

In preferred embodiments of reducing the phase transition temperature of a cell, the cell is grown in a culture media containing supplemental lipids, such as cholesterol and lipids comprising relatively short chain (C12-C16) fatty acids; more preferably unsaturated. For example, the cells can be grown in a standard media for the cell, but supplemented to contain at least 1 mg/L, 5 mg/L, 10 mg/L, 25 mg/L, 50 mg/L, 100 mg/L, 200 mg/L, 400 mg/L, or more of cholesterol and/or lipids comprising one or more C12 to C16 unsaturated fatty acid. It is preferred that the fatty acids of the lipids be present in amounts greater than for other fatty acids (such as, C18-C22 saturated fatty acids). Optionally, to increase membrane phase transition temperatures, the host cell can be grown in media with a lower variety of different classes and/or subclasses of lipids. To help reduce the membrane phase transition temperature of the cells, it is preferred that unsaturated C12-C16 fatty acids be present in culture media in amounts at least 10 mole percent, at least 15%, at least 20%, at lease 30%, at least 40% or more of an individual media lipid type or of the total media lipids Optionally, to decrease membrane phase transition temperatures; the cell can be grown in media with a greater variety of different classes and/or subclasses of lipids. In a preferred embodiment of adjusting the phase transition temperature of a virus membrane, the virus is cultured using host cells treated as described above.

In preferred embodiments of increasing the phase transition temperature of a cell, the cell is grown in a culture media containing supplemental lipids, such as sphingolipids and other lipids comprising relatively long chain (C18-C22) fatty acids; more preferably saturated; more preferably with a limited number of different lipids. For example, the cells can be grown in a standard media for the cell, but supplemented to contain at least 1 mg/L, 5 mg/L, 10 mg/L, 25 mg/L, 50 mg/L, 100 mg/L, 200 mg/L, 400 mg/L, or more of sphingomyelin and/or other lipids comprising one or more C18 to C22 saturated fatty acid. It is preferred that the fatty acids of the lipids be present in amounts greater than for other fatty acids (such as, C12-C16 unsaturated fatty acids). To help reduce the membrane phase transition temperature of the cells, it is preferred that unsaturated C18-C22 fatty acids be present in culture media in amounts at least 10 mole percent, at least 15%, at least 20%, at lease 30%, at least 40% or more for an individual media lipid type or for the total media lipids. In a preferred embodiment of increasing the phase transition temperature of a virus membrane, the virus is cultured using host cells grown in media as described above. Optionally, the phase transition temperature of a cell or virus membrane can be increased by exposure to sphingomyelinase and/or cyclodextrins.

### Systems for Modulating the Membrane Lipids of a Cell or Virus

The present invention includes systems for modification of lipid constituents in the membrane of a cell or virus. The systems generally include, e.g., one or more cells in an appropriate growth media, the media having supplemental phospholipids, cholesterol and/or sphingomyelin. The system can include a virus capable of infecting the cells.

Systems and methods for manipulation of cell and virus lipids include supplementation of media wherein the identified ingredient is absent or deficient. Supplementation can optionally include addition of the identified lipids to standard media, well known in the an with known or unpublished ingredients. The present inventions envision modification of cell and virus lipids by making the identified modifications by addition of (or reformulation to remove) the specific lipids taught herein. For example, certain standard media, although possibly containing some amount of the lipid for modification, can be modulated for the identified lipids to affect the lipid content of a cell or virus. For example, the following media can be lipid modulated, as taught herein, to modify host cell and/or virus lipid compositions: 293 Cell Media, Adipocyte/Preadipocyte Media, Amniotic Fluid Cell Media, Astrocyte Media, Basal Medium Eagle, Blood & Bone Marrow Cell Media, CHO Cell Media, Chondrocyte Media, Click's Media, CMRL 1066 Media, D-MEM w/o Glutamine, D-MEM/F12 Media, D-MEM w/Glutamine, Endothelial Cell Media, Fibroblast Medium, Gene Therapy Media, G-MEM Media, Ham's F-10 Media, Ham's F-12 Media, Hepatocyte Media, Hybridoma Media, IMEM Richter's Medium, Iscove's Media, Keratinocyte Media, Liebovitz L-15 Media, McCoy's 5A Media, MCDB 131 Media, MDCK Media, Medium 199, MEM Alpha Media, MEM Spinner Media, MEM w/Glutamine, MEM w/o Glutamine, NCTC-109 Media, Neuronal Cell Media, Oligodendrocyte Media, Osteoblast Media, RPMI 1640 Media, Smooth Cell Medium, Transfected Cell Selection & Cloning Media, VERO Cell Media, Williams' Media, Murashige & Skoog basal salt solution, Chu (N6) medium, DKW/Juglans Basal Salt Mixture, McCowen's Woody Plant Media, Guillard's (F/2) Marine Enrichment Basal Salt, and the like (see, e.g., products of Invitrogen, Sigma-Aldrich, and the like).

The cells, media and virus can be contained in a culture container, such as a fermenter, T-flask, Petri dish, flask, stir flask, and the like. The systems can include sub system components to provide a stable incubation temperature, e.g., from about 10° C. to about 40° C., or about 37° C.

In particular embodiments of the systems, the sphingomyelin can comprise C16:0 fatty acids, C18:0 fatty acids, C182n6 and/or C181n9 fatty acids. For example the media sphingomyelin can contain (esterified) 25 mole percent or more palmitate (C16:0), 20 mole percent or more stearate (C18:0) and/or 40 mole percent or more oleate (C18:1n9).

In another aspect of the methods and systems, the media for culture of virus host cells comprises supplemental (as compared to incidental or standard media amounts) phosphatidylserine and/or phosphatidylinositol. For example, the cells can be grown in a standard media for the cell, but supplemented to contain at least 1 mg/L, 5 mg/L, 10 mg/L, 25 mg/L, 50 mg/L, 100 mg/L, 200 mg/L, 400 mg/L, or more of phosphatidylserine and/or phosphatidylinositol. It is preferred that the fatty acids of these phospholipids be predominantly C16:0, C18:0, C181n9, and/or C18:2n6. Most preferred phospholipids supplements comprise one or more C18:2n6 fatty acids. It is preferred that phosphatidylserine and/or phosphatidylinositol be present in culture media in amounts at least 10 mole percent, at least 15%, at least 20%, at lease 30%, at least 40% or more for of the phospholipids or total lipids in the media.

In preferred embodiments, the cells are animal cells. Preferably, the cells are bird or mammal cells. More preferably, the cells are human cells, monkey cells, ape cells, chicken cells. Most preferably, the cells are chicken allantoic sac (CAS) cells, chicken embryo kidney (CEK) cells, canine kidney cells, Vero cells, lymphocytes, MDCK cells, MA-104 cells, CHO cells, HeLa cells, 293 cells, NSO cells, and U937 cells.

In preferred embodiments, the virus is an enveloped virus, such as members of the Baculoviridae, Herpesviridae, Iridoviridae, Poxviridae, Cystoviridae, Coronaviridae, Flaviviridae, Togaviridae, Arterivirus, Retroviridae, Orthomyxoviridae, Filoviridae, Paramyxoviridae, Rhabdoviridae, Arenaviridae, and Bunyaviridae families. Most preferred viruses include the various Influenza viruses, human immunodeficiency (HIV) viruses, paramyxovirus (such as respiratory syncytial virus, parainfluenza virus, metapneumovirus), and papovavirus (such as human papilloma virus).

Culture of viruses in the systems of the invention can allow control over certain characteristics of the progeny virions. For example, culture of host cells in the supplemented (modulated) media can provide a higher titer than for virus cultured host cells grown on unsupplemented media, provide a longer shelf life for the virus, provide a higher immunogenicity for the virus, and/or provide a modified membrane Tg for the virus.

In other aspects of the systems and methods, the lipid content of host cells (e.g., CAS cells) and/or virions can be influenced by feeding an animal providing the host cells with a diet enriched for the lipids identified herein as providing advantages. For example, the animal can be fed a diet enriched in lipids, such as sphingomyelin, phospholipids, and/or cholesterol esters, comprising C16:0, C18:0, C181n9, and/or C18:2n6 fatty acids.

### Examples

The following examples are offered to illustrate, but not to limit the claimed invention. One of skill will recognize a variety of parameters that can be altered while obtaining substantially similar results.

Phase transition measurements of Influenza viral envelopes revealed a relatively rigid membrane structure, evidence of high concentrations of high melting lipids, and abundant cholesterol and sphingolipids on viral envelope. The question considered was—are certain lipid classes preferentially or selectively enriched in virions as compared to the host cell on which they grow? We studied the types and subclasses of lipids enriched in viruses; the differences in lipid composition of host cells with different levels of permissivity, and effects of cell culture media supplementation with “preferential” lipid components.

We found that lipid composition differences between and among host cells and virions correlated with characteristics useful in altering the productivity and efficacy of cell and virus products. For example, lipid composition analysis indicated enhanced levels of sphingomyelin and cholesterol were over represented in virus envelopes as compared to the host cells that it is generated from. We noted significant differences between viruses and cells with respect to sphingomyelin subtypes and cholesterol subtypes. Sphingomyelin 16:0 was found in high concentrations in virus while 18:0 was higher in permissive cells but reduced (perhaps preferentially) in virus. Cholesterol esters with 18:1n9 chain-length were preferentially enriched in cells, while 16:0 was enriched in viruses. Vero cells (low permissivity for Influenza) contain lower sphingomyelin and significantly lower 18:1n9 cholesterol ester content than allantoids sac cells which have a high permissivity to flu infection.

### Example 1

### FTIR Microscopy of Membrane Phase Transitions

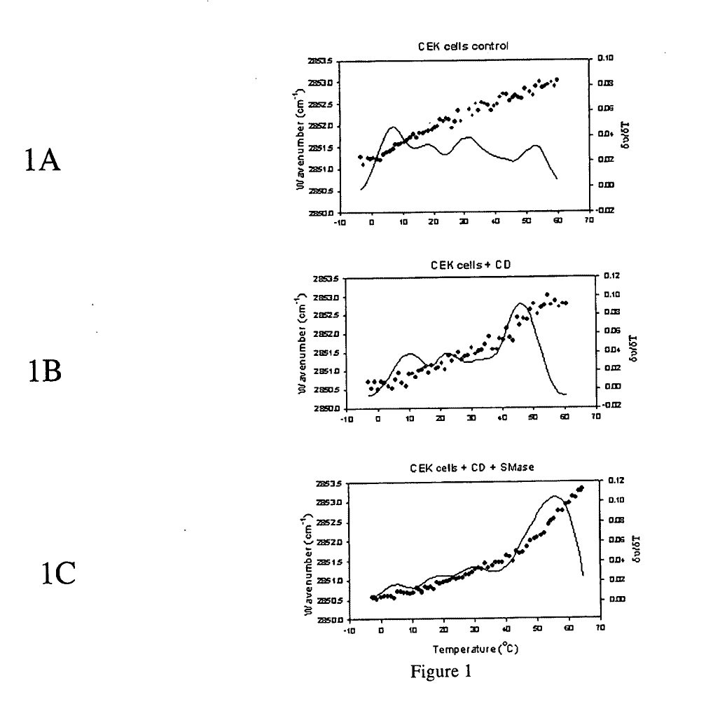

As shown in FIGS. 1A to 1C, lipid mobility of membranes was influenced by the cholesterol and sphingomyelin content of the membranes. The FTIR detected symmetric —CH2 stretching vibrations at ˜2850 cm⁻¹, and to monitor vibrations of fatty acyl side chains of phospholipid tail groups. Wave number vs. temperature was plotted to identify membrane phase changes. Fluid phase high lipid mobility phase changes to gel-crystalline phase changes was detectable. Transition to higher wave numbers were found to indicate a higher order (or more rigid) membrane lipid environment.

The hydrophobic pocket of cyclodexrins has the ability to sequester cholesterol from membranes. When membrane cholesterol content was reduced by cyclodextrin treatment, as compared to the control of FIG. 1A, lipid mobility was reduced and the lipids found more ordered, as shown in FIG. 1B. As shown in FIG. 1C, the membrane melting point and lipid order was further increased by treatment of the membranes with sphingomyelinase, thus converting membrane sphingomyelin to ceramide. Further, these experiments showed that CEK cells, highly permissive for Influenza infection, contain abundant cholesterol and sphingolipids amenable of modulation to change the membrane character.

In one aspect, it is envisioned that FTIR analyses can be used to predict permissivity of host cells. For example, certain FTIR profiles and/or responses to treatments can correlate to permissivity. It is expected that modulation of host cell media can provide similar changes in membrane lipid composition and membrane characteristics.

### Example 2

### FTIR Analysis of Treated Virion Membranes

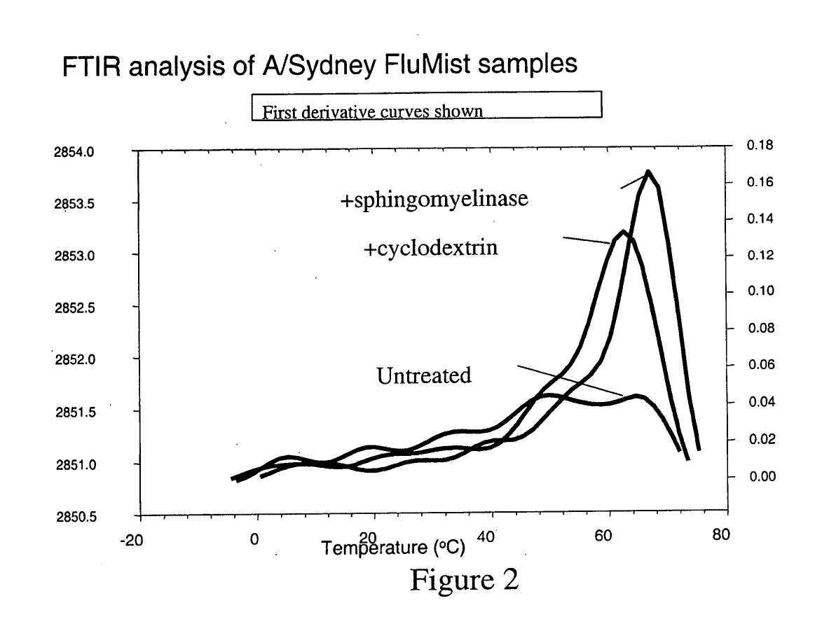

The order and lipid mobility of virus membranes were also found to be affected by changes in the lipid content. For example, as shown in FIG. 2, modification of the cholesterol content of the virus membrane (e.g., by cyclodextrin treatment) can change the mobility and phase transition characteristics. Moreover, conversion of membrane sphingomyelin to ceramide can also change the character of the membrane, as detected by FTIR. These experiments show that, as with permissive host cells, the virus has a relatively large amount of cholesterol and sphingomyelin. Virus membranes are shown to be enriched with cholesterol and sphingomyelin, even over the membranes of their most permissive host cells.

In one aspect, it is envisioned that FTIR analyses can be used to detect changes in a virus, e.g., with storage. For example, degradation or depletion of certain membrane lipid components can be correlated to changes in the FTIR profile.

From these experiments, it was observed that the flu envelope is relatively rigid, as evidenced by the magnitude of change in wave numbers. Flu likely contains high concentrations of cholesterols and sphingolipids. A large phase transition is detectable for A/Sydney at ˜15° C., but less so for B/Harbin. Such a phase transition can be used to design a drying cycle in a formulation drying process, e.g., for a virus vaccine. As for HIV, the Influenza envelope is derived from membrane lipid rafts or microdomains atypical of the average host membrane.

### Example 3

### Membrane Compositional Analysis Including Lipid Subtypes

Thin layer chromatography (TLC) and gas chromatographic methods were used to identify membrane lipids of 10 classes and 40 subclasses. The classes included; phosphatidylcholines, phosphatidylethanolamines, phosphatidylserine/inositols, sphingomyelins, cholesterol esters, free fatty acids, diacylglycerides, triacylglycerides, cardiolipins and lysophosphatidylcholines. Within the classes, the lipids were further characterized according to, e.g., what fatty acids are esterified to the lipid structure. The detection limit was typically about 1 nMole, and the assay variability was in the range of less than 10% run to run.

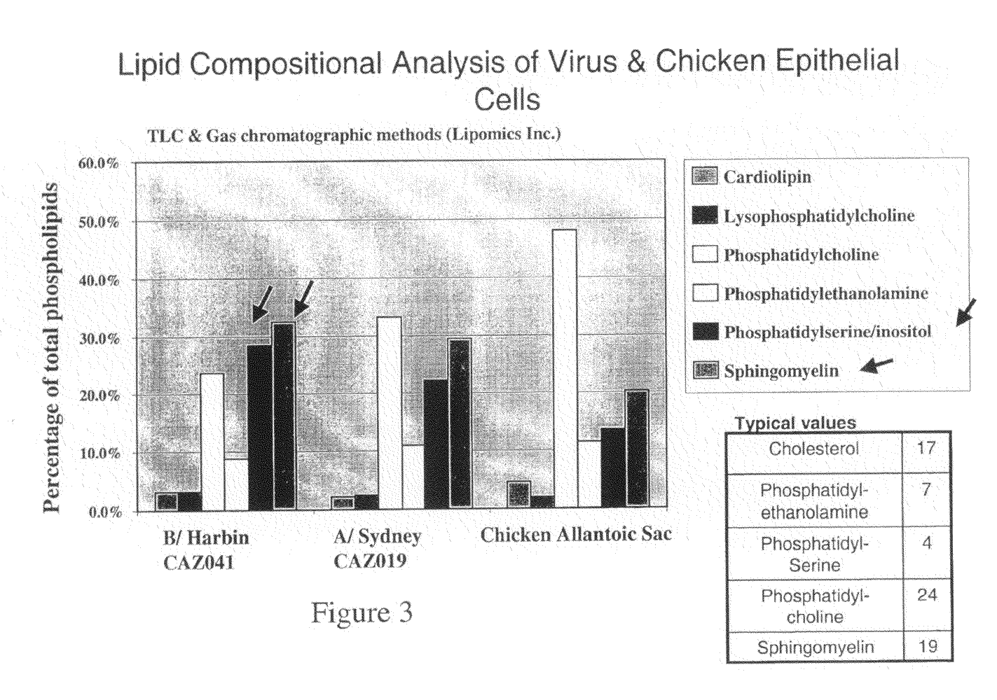

As shown in FIG. 3, among other things, the virus envelope membranes are enriched with sphingomyelin and glycolipids (phosphatidylserine/inositol), as compared to the host CAS cell membrane.

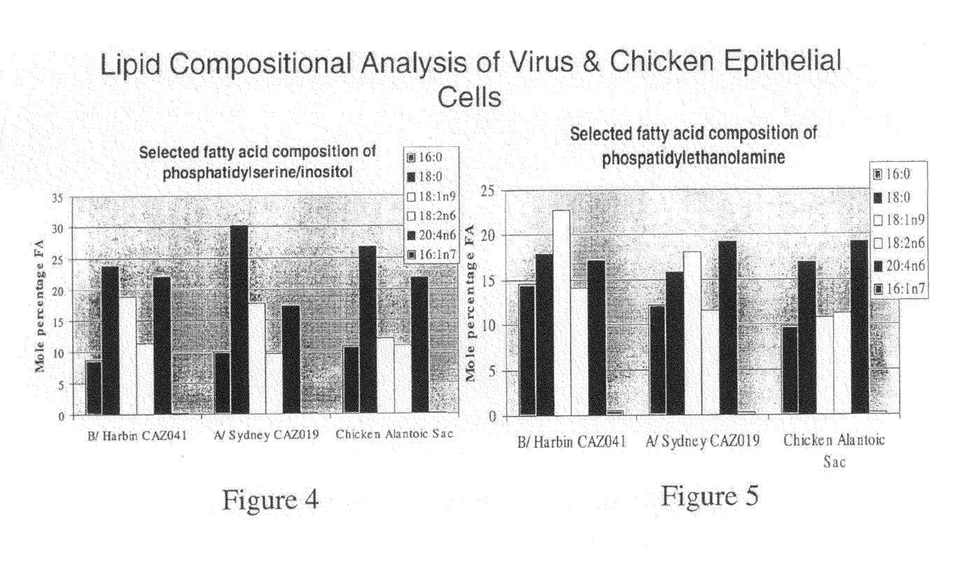

As shown in FIGS. 4 and 5, significant differences appear in lipid content subtypes between the host cell and progeny viruses. For example, for both phosphatidylethanolamine and phosphatidylserine/inositol, there appears to be an enrichment of C18:1n9 fatty acids in the viruses over the host.

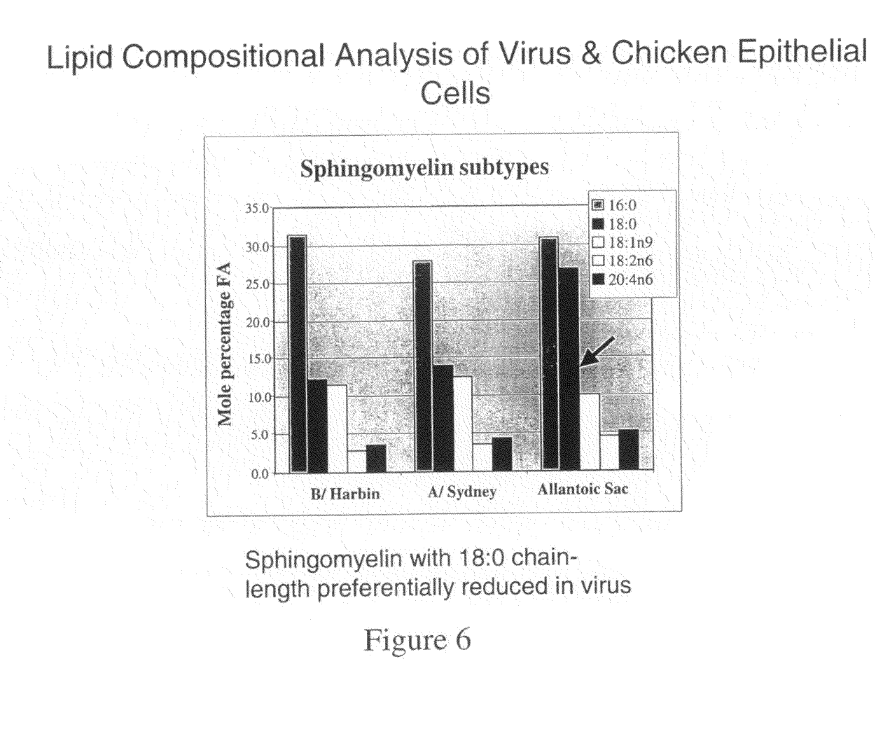

As shown in FIG. 6, There is a substantial enrichment of sphingomyelin subtypes with C18:0 fatty acids in the viruses over host cell levels.

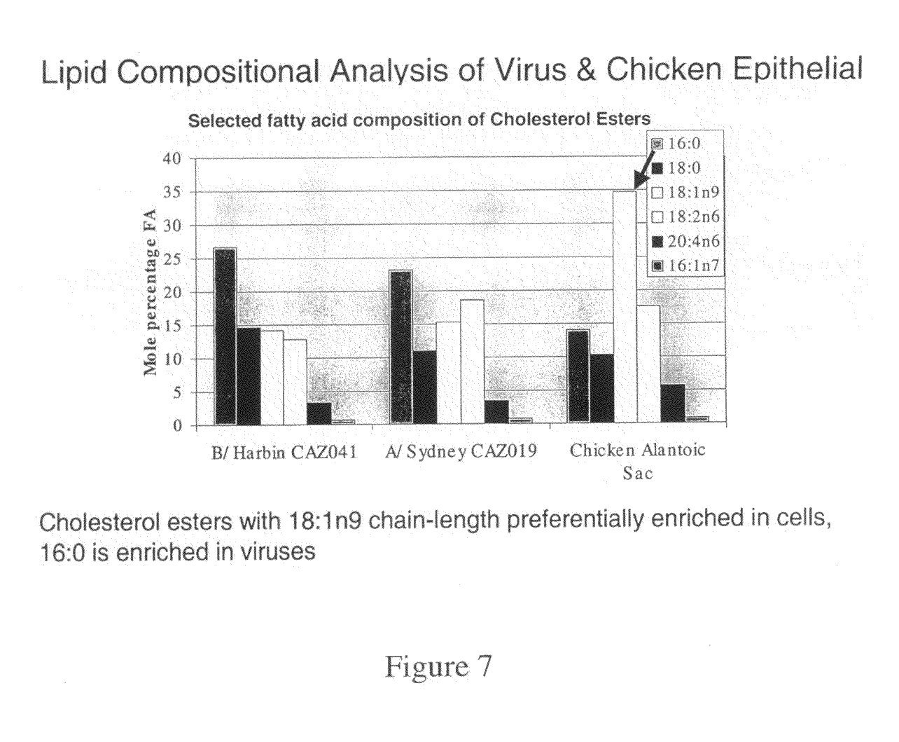

As shown in FIG. 7, permissive CAS cells are significantly enriched for cholesterol esters comprising C18:1n9 over progeny viruses.

### Example 4

### Membrane Compositional of Permissive and Non-Permissive Cells

A series of analyses were performed on a selection of host cells with varying degrees of permissivity of infection by viruses. Trends were identified correlating particular membrane lipids with sensitivity to infection by the viruses.

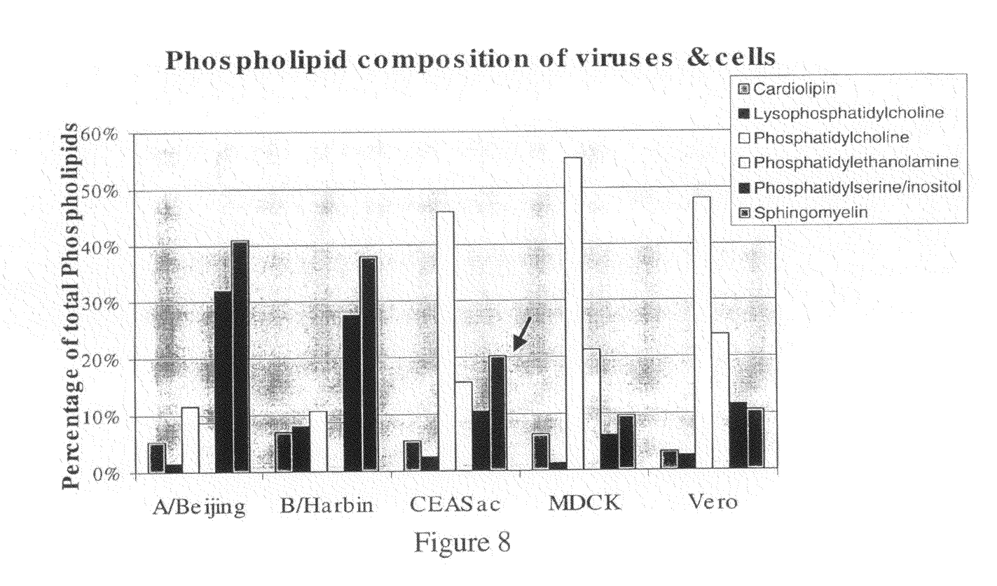

For example, Vero cells are relatively less permissive for infection by Influenza viruses, MDCK cells are somewhat more permissive and CAS cells are most permissive. As shown in FIG. 8, sphingomyelin is substantially more prominent in the permissive cells.

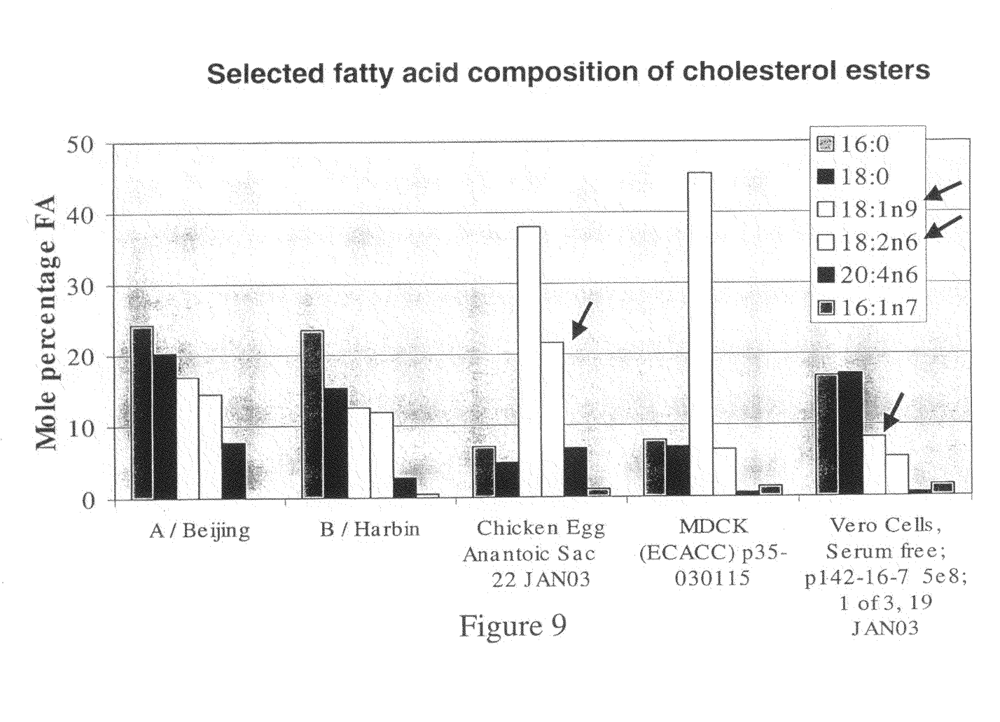

With regard to esters if cholesterol, C18:1n9 and C182n6 fatty acids are significantly more prominent in permissive cells than less permissive cells, as shown in FIG. 9.

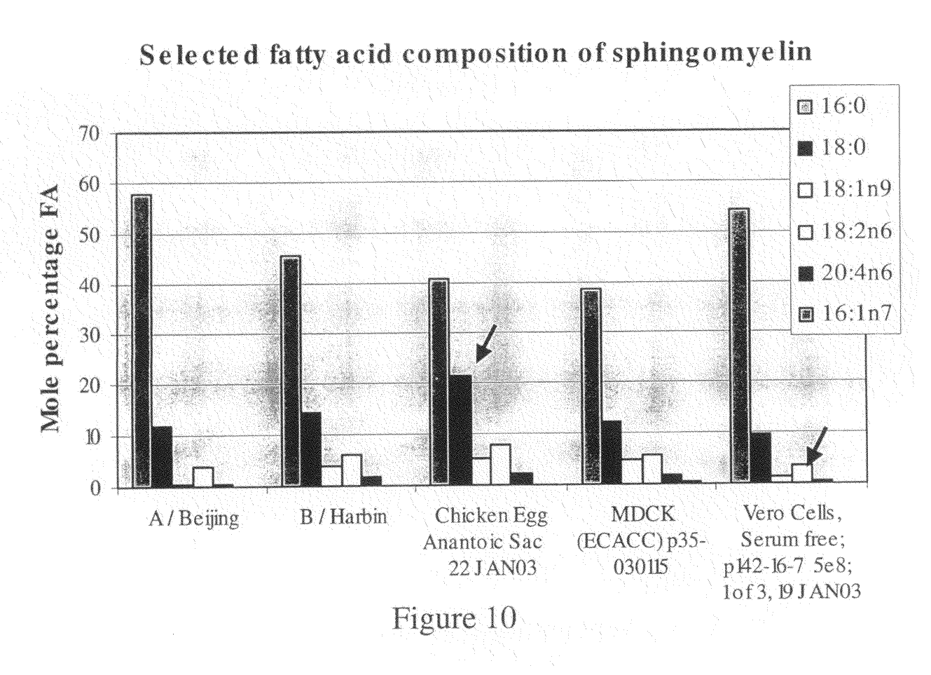

With regard to sphingomyelin subtypes, as shown in FIG. 10, there appears to be a correlation between permissivity and the abundance of C18:0 containing sphingomyelin.

In summary, adjustments of cell and virus membrane lipids hold the promise of influencing permissivity of host cells and the yield of virus cultures. Phase transition measurements suggest Influenza viral envelopes are very rigid and composed of high melting lipids. Lipid composition analysis have confirmed increased levels of sphingomyelin and cholesterol in the virus envelope. Significant differences were observed between virus and cells with respect to sphingomyelin subtypes and cholesterol subtypes. Vero cells contain lower sphingomyelin and significantly lower 18:1n9 cholesterol ester content than more permissive allantoic sac cells.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A method of modifying a virus lipid composition, the method comprising:
modulating levels of sphingomylein or cholesterol in a culture media for culturing a type of host cell suitable for the virus; and,
infecting the host cell with the virus;
thereby providing a virus with a different lipid composition than a control virus cultured on the host cell type grown on the culture media without modulation of the sphingomylein or cholesterol levels.

2. The method of claim 1, wherein the virus is selected from the group consisting of: a retrovirus, a paramyxovirus, respiratory syncytial virus, parainfluenza virus, metapneumovirus, an orthomyxovirus, influenza virus, a papovavirus, human papilloma virus, filovirus, poxvirus, herpesvirus, hepadnavirus, a rhabdovirus, rabies, a coronavirus, SARS, togavirus, arenavirus, and bunyavirus.

3. The method of claim 1, further comprising changing a viral membrane phase transition temperature by the modulating the levels of the sphingomylein or cholesterol in the media.

4. The method of claim 3, further comprising holding the virus in a liquid preservative formulation at the transition temperature of the lipid membrane.

5. The method of claim 4, further comprising drying the virus by a method selected from the group consisting of: freeze drying, freeze foam drying, foam drying, spray freeze drying, spray drying, high pressure spray drying, and supercritical spray drying.

6. The method of claim 1, wherein the total levels of sphingomylein and cholesterol are modulated to greater than 20 mg/L or less than 0.01 mg/L in the media.

7. The method of claim 1, further comprising modulating the sphingomylein or cholesterol levels until the immunogenicity of the virus is altered.

8. The method of claim 1, further comprising modulating the sphingomylein or cholesterol levels in the media until the permissivity of the host cells is increased for the virus.

9. The method of claim 8, wherein said modulating comprises increasing sphingomylein levels or increasing cholesterol comprising 9-octadecenoic acid (C18:1n9).

10. The method of claim 1, further comprising modulating the levels of sphingomylein or cholesterol in the media to a point where the production of the virus from the cell is increased.

11. The method of claim 1, wherein the sphingomylein or cholesterol comprises fatty acids selected from the group consisting of: palmitate (C16:0), stearate (C18:0), and oleate (C18:1).

12. The method of claim 1, further comprising adding phosphatidylserine or phosphatidylinositol to the media, thereby providing an enhanced yield of progeny virions.

13. An Influenza virion comprising an increased proportion of membrane cholesterol or sphingomylein as compared to a control influenza virion grown in the allantoic sac of a chicken egg.

14. The virion of claim 13, wherein the increased proportion results from culture of the virion on host cells grown in media containing 20 mg/L or more of sphingomylein or cholesterol.

15. The virion of claim 13, wherein the mole percentage fatty acids in the membrane of sphingomylein palmitate (C16:0) is increased by at least 10% over that of the control virion or the cholesterol palmitate (C16:0) or stearate (C18:0) is increased by at least 10% over that of the control virion.

16. A system for culture of a virus, the system comprising:
a host cell permissive for the virus;
the cell in a cell culture media suitable for growing the cell; and,
cholesterol or sphingomylein;
whereby addition of the cholesterol or sphingomylein to the media modulates the permissivity, increases yield of the virus from the cell, increased stability of the virus in storage or changes the membrane phase transition temperature of the virus.

17. The system of claim 16, wherein the additional cholesterol or sphingomylein comprises more than 25 mole percent palmitate (C16:0), more than 20 mole percent stearate (C18:0) or more than 40 mole percent oleate (C18:1) than in the media before modulation by the addition of the cholesterol or sphingomylein.

18. The system of claim 16, wherein the virion is selected from the group consisting of: a retrovirus, a paramyxovirus, respiratory syncytial virus, parainfluenza virus, metapneumovirus, an orthomyxovirus, influenza virus, a papovavirus, human papilloma virus, filovirus, poxvirus, herpesvirus, hepadnavirus, a rhabdovirus, rabies, a coronavirus, SARS, togavirus, arenavirus, and bunyavirus.

19. The system of claim 16, wherein the cell is selected from the group consisting of: chicken allantoic sac (CAS) cells, chicken embryo kidney (CEK) cells, canine kidney cells, Vero cells, lymphocytes, MDCK kidney cells, MA-104 cells, CHO cells, HeLA cells, 293 cells, NSO cells, and U937 cells.

20. The system of claim 16, wherein the media comprises phosphatidylserine or phosphatidylinositol.

21. A method of providing an influenza virus with a modified membrane lipid composition, the method comprising:
feeding a chicken a diet rich in cholesterol or sphingomylein;
collecting one or more fertile eggs from the chicken;
inoculating the allantoic sacs of the one or more eggs with an influenza virus; and,
harvesting influenza viruses from the one or more allantoic sacs.

22. The method of claim 21, wherein the cholesterol or sphingomylein comprise more than 25 mole percent palmitate (C16:0), more than 20 mole percent stearate (C18:0) or more than 40 mole percent oleate (C18:1).

23. The method of claim 21, wherein the diet rich in cholesterol or sphingomylein comprises at least 1 gram of total cholesterol or total sphingomylein per chicken per day.

24. The method of claim 21, wherein the cholesterol or sphingomylein of the diet is modulated at least until viruses provided comprise a characteristic selected from the group consisting of: a higher titer than for virus cultured on eggs from control chickens not fed a diet rich in cholesterol or sphingomylein, a longer shelf life than for virus cultured on eggs from control chickens not fed a diet rich in cholesterol or sphingomylein, a higher antigenicity than for virus cultured on eggs from control chickens not fed a diet rich in cholesterol or sphingomylein, and a modified membrane phase transition compared to a membrane from a virus cultured on eggs from control chickens not fed a diet rich in cholesterol or sphingomylein.
